(19) United States
(12) Patent Application Publication
(10) Pub. No.: US 2022/0160944 A1
(43) Pub. Date: May 26, 2022

(54) CONNECTOR FOR VALVE IMPLANT

(71) Applicant: Mohammed Ibn khayat Zougari, Windsor (CA)

(72) Inventor: Mohammed Ibn khayat Zougari, Windsor (CA)

(21) Appl. No.: 17/533,093

(22) Filed: Nov. 22, 2021

Related U.S. Application Data
(60) Provisional application No. 63/196,357, filed on Jun. 3, 2021.
(60) Provisional application No. 63/116,476, filed on Nov. 20, 2020.

(51) Int. Cl. A61M 1/36; A61F 2/24

(52) U.S. Cl. CPC A61M 1/3655; A61F 2002/061; A61M 1/3661; A61F 2/2476

(57) ABSTRACT

A connector assembly for an arteriovenous (AV) graft according to an example of the present disclosure includes, among other possible things, a connector having a first segment arranged a long a first axis having first and second opposing ports and a second segment arranged along a second axis having a third port, each of the first and second ports configured to be joined to first and second portions of an artery or a vein, respectively. At least one ring is configured to connect the first and second ports to the first and second portions of the artery or the vein by applying force about substantially the circumferential extent of the first segment of the connector and the first and second portions of the artery or the vein to join the first and second ports to the first and second portions of the artery or the vein, respectively. An AV graft assembly and method of implanting a connector for an AV graft are also disclosed.

### CROSS-REFERENCE TO RELATED APPLICATIONS

This application claims priority to U.S. Provisional Application 63/196,357, filed Jun. 3, 2021; and U.S. Provisional Application 63/116,476, filed Nov. 20, 2020; which are herein incorporated by reference in their entireties.

### BACKGROUND

Hemodialysis is a medical procedure that requires vascular access (that is, access to a patient's vascular system, including veins and arteries) via an AV graft, which is a biocompatible tube that links a patient's artery and vein. The tube has access points for access from outside of the patient's body. Connecting the graft to the patient's veins and arteries can pose certain challenges, which can cause complications for the patient.

### SUMMARY

A connector assembly for an arteriovenous (AV) graft according to an example of the present disclosure includes, among other possible things, a connector having a first segment arranged a long a first axis having first and second opposing ports and a second segment arranged along a second axis having a third port, each of the first and second ports configured to be joined to first and second portions of an artery or a vein, respectively. At least one ring is configured to connect the first and second ports to the first and second portions of the artery or the vein by applying force about substantially the circumferential extent of the first segment of the connector and the first and second portions of the artery or the vein to join the first and second ports to the first and second portions of the artery or the vein, respectively.

An arteriovenous (AV) graft assembly according to an example of the present disclosure includes, among other possible things, a first connector, the first connector having a first segment arranged a long a first axis having first and second opposing ports and a second segment arranged along a second axis having a third port, each of the first and second ports configured to be joined to first and second portions of an artery, respectively. The AV graft assembly also includes a second connector, the second connector having a first segment arranged a long a first axis having first and second opposing ports and a second segment arranged along a second axis having a third port, each of the first and second ports configured to be joined to first and second portions of an vein, respectively. The AV graft assembly also includes an AV graft joined to the third port of each of the first and second connectors.

A method of implanting a connector for an AV graft according to an example of the present disclosure includes, among other possible things, forming an opening in an artery or a vein between first and second portions of the artery or vein, arranging a connector in the opening, the connector having a first segment arranged a long a first axis having first and second opposing ports and a second segment arranged along a second axis having a third port, and joining the first and second portions of the artery or vein to the first and second ports.

### DESCRIPTION OF THE FIGURES

The various features and advantages of the disclosed examples will become apparent to those skilled in the art from the detailed description. The figures that accompany the detailed description can be briefly described as follows:

FIG. 1 schematically illustrates an arteriovenous (AV) graft.

FIGS. 2a-b schematically illustrates an AV graft connected to a vein and an artery by a connector.

FIGS. 3a-b schematically illustrates a perspective view of example connectors.

FIGS. 4a-b schematically illustrate an example ring for joining the connector of FIGS. 2a-b and 3a-b with a vein or artery.

FIG. 5 schematically illustrates the connector of FIGS. 3a-3b inserted into a vein or artery with a seal.

FIG. 6 schematically shows example connectors of FIGS. 1-5 integral with an AV graft.

FIGS. 7a-b schematically show example connectors in a low profile operating mode.

### DETAILED DESCRIPTION

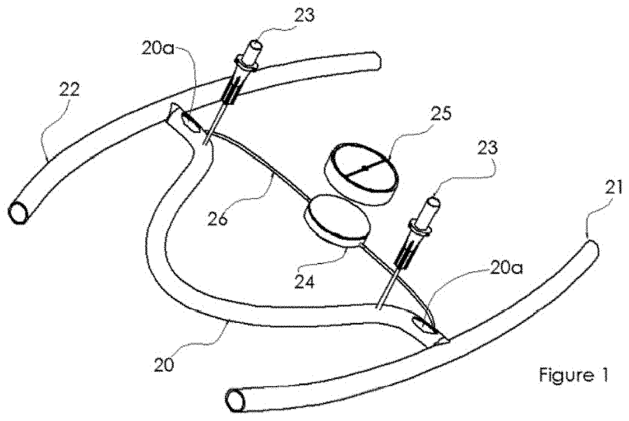

An arteriouvenous (AV) graft 20, shown in FIG. 1, is a biocompatible tube that links a patient's artery 21 and vein 22. The AV graft 20 provides vascular access for hemodialysis. The AV graft 20 has access points 23 for access from outside of the patient's body, to connect to a hemodialysis machine. In some examples, the AV graft 20 has a valve for controlling blood flow through the graft, such as a balloon valve 20a. The example balloon valve 20a in an inflated state blocks blood flow through the AV graft, and in a deflated state allows blood to flow through the AV graft 20a. The example AV graft 20 also has access points to an active fluid line 26, which includes a valve 24 (e.g., a driven element). An actuator 25 (e.g., a driving element) actuates the valve externally (from outside the body). The active fluid line 26 receives active fluid, such as saline solution. The valve 24 selectively controls the flow of active fluid, which in turn controls blood flow through the AV graft 20. That is, the valve 24 can allow blood flow through the AV graft 20 during the hemodialysis procedure, and disallow blood flow at all other times via the actuator 25. In this way, blood flow between the artery 21 and vein 22 is only allowed when necessary to facilitate hemodialysis, reducing the risk of complications from the unnatural diversion of blood. Though a valve for an AV graft is contemplated, it should be understood that the present disclosure is not limited to AV grafts and can be used in other applications as well. An example valve and actuation mechanism is described in U.S. Pat. No. 10,610,633, which is hereby incorporated by reference in its entirety.

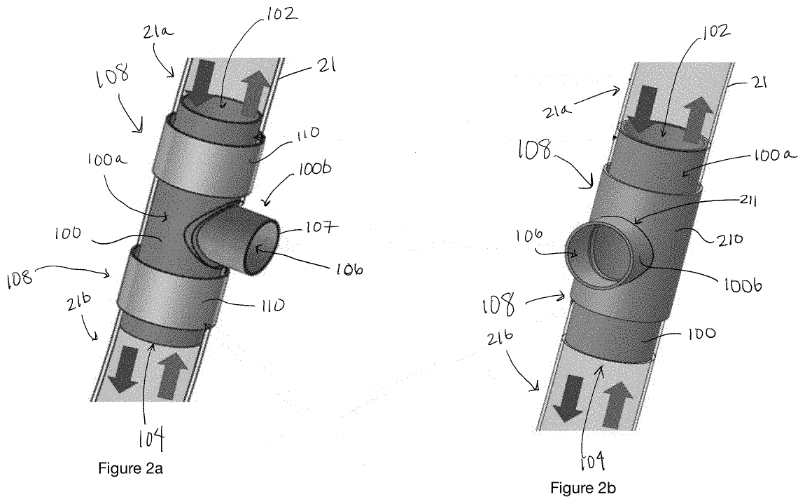

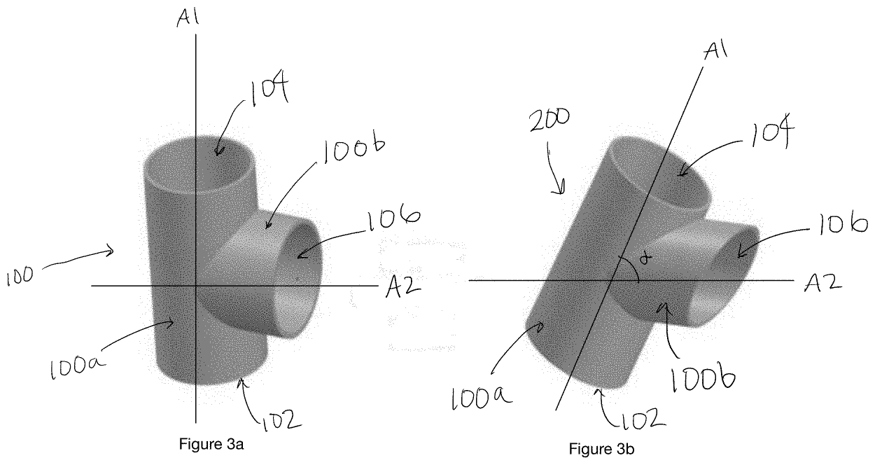

Implanting an AV graft requires fluidly connecting the AV graft 20 to a patient's artery 21 and vein 22. FIGS. 2a-b schematically show a T-branch connector 100 fluidly connecting an AV graft 20 to an artery or vein 21/22. FIG. 3a shows a perspective view of the connector 100. The connector 100 has a first segment 100a having opposed ports 102/104 arranged along a first axis A1. A second segment 100b has a third port 106 and is arranged along a second axis A2. In the example of FIG. 3a, the first and second axes A1/A2 are generally perpendicular to one another. However, in other examples, such as the example of FIG. 3b, an alternate connector 200 has an angle α between the first and second axes A1/A2 that is between 70 and 110 degrees. In general, the connector 100/200 has a “T” shape.

The first and second ports 102/104 are configured to be joined with an artery or vein 21/22 to continue fluid flow through the artery or vein 21/22, and the third port 106 is configured to be joined to the AV graft 20. The join can be by stitches/sutures, or biocompatible tape or glue, in some examples. In this way, the connector 100/200 enables a fluid connection between the artery 21, the vein 22, and the AV graft 20. The connector 100/200 has a smooth interior surface that does not substantially interfere with blood flow through the artery or vein 21/22. In one example, each of the ports 102/104/106 have smooth chamfered edges 107.

In order to receive the connector 100/200, an opening 50 is formed in the artery 21. In one example, the artery 21 is bisected, e.g., fully separated into two portions 21a/21b, with the opening 50 between them. The connector 100/200 is arranged between the two portions 21a/21b and the portions 21a/21b are joined to the ports 102/104, respectively, as discussed above, such that the ports 102/104 enable continued fluid flow through the artery 21 while also providing fluid communication with the third port 106 and the AV graft 20.

The connector 100/200 may be situated such that the ports 102/104 extend at least partially into the portions 21a/21b of the artery 21 at joins 108. In another example, the connector 100/200 may be situated such that the portions 21a/21b of the artery 21 extend into the ports 102/104 at the joins 108.

In one example, the ports 102/104 can be joined to the portions 21a/21b of the artery 21 by rings 110. The rings 110 can be used in place of or in conjunction with sutures, tape, or glue as discussed above. In the example of FIG. 2a, there are two rings 110, one associated with the join 108 at each port 102/104. The rings 110 apply force about substantially the circumferential extent of the segment 100a of the connector 100/200 and portions 21a/21b at the joins 108 to substantially seal the portions 21a/21b against the connector 100/200. In some examples, the rings 110 can allow the connector 100/200, and thus the AV graft 20, to be secured to the artery 21 without the use of sutures, which can improve healing times and reduce the risk of complications.

FIG. 2b shows another example ring 210. In this example, a single ring 210 is fitted around the segment 100a of the connector 100/200. In this example, the ring 210 includes an opening 211 which is configured to receive the segment 100b of the connector 100/200. The ring 210 in this example still provides adequate force to join the connector 100/200 to the portions 21a/21b of the artery 21 as discussed above.

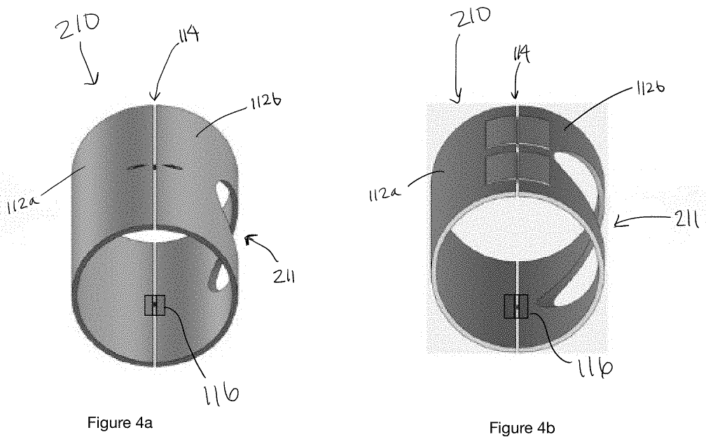

FIGS. 4a-b show perspective views of the example ring 210. The ring 210 includes two segments 112a/112b connected by a hinged connection 114. The hinged connection 114 can be spring loaded as is known in the art, in some examples, to urge the segments 112a/112b towards one another and increase the force provided by the ring 210. The ring 210 may also include a closure or lock 116 opposite the hinged connection 114. Any type of closure 116 known in the art can be used, such as a tongue and groove closure or a magnetic closure. Additionally or alternatively, biocompatible tape or glue can also be used at the closure 116.

The ring 110 can have the same segments, hinged connection and closure as the ring 210 as discussed above.

The ring 110/210 can be formed as a unitary component, e.g., the segments 112a/112b and the hinged connection 114 are integral with one another. In other examples, however, the segments 112a/112b and hinged connection 114 are formed separately and assembled with one another.

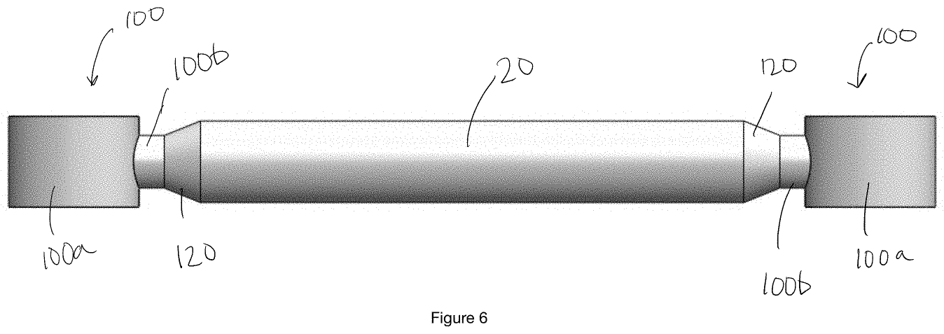

The third port 106 can be connected to the AV graft 20 in any known biocompatible way, such as by a biocompatible glue or tape, or by another ring 110 as discussed above. In another example, shows in FIG. 6, two connectors 100/200 are formed integrally with an AV graft 20, e.g., the connectors 100/200 and AV graft 20 are a unitary structure.

Also shown in FIG. 6 is an optional nozzle 120 extending from the port 106 of segment 100b of the connector 100/200. It should be understood that the nozzle 120 could be used with a connector 100/200 that is formed separated from the AV graft 20 or with connectors 100/200 that are integral with the AV graft 20.

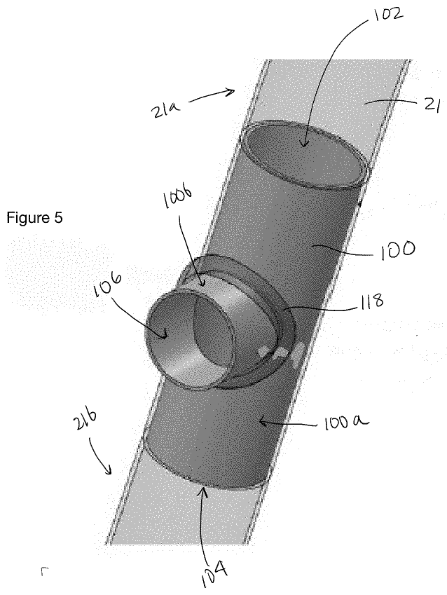

As discussed above, in one example, the artery 21 is bisected to form the opening 50. In other examples, the artery 21 punctured or incised to form the opening 50, but such that the portions 21a/21b remain at least partially connected. The opening 50 may be 2-3 mm in length, in some examples. In this example, pictured in FIG. 5, the connector 100/200 is positioned inside the artery 21, with the segment 100b protruding from the opening 50 for connection with the AV graft 20. The segment 100b has a diameter sized to fit through the opening 50. Though not shown in FIG. 5, the rings 110/210 discussed above can also be used around the joins 108 at ports 102/104 in this example.

In the example of FIG. 5, a seal 118 is arranged around the opening 50. The seal 118 reduces leakage of blood at the opening 50. The seal is annular in shape and can be made from any biocompatible material such as biocompatible tape or glue.

Optionally, seals 118 can also be included around joins 108 any of the ports 102/104/106 in any of the examples discussed above.

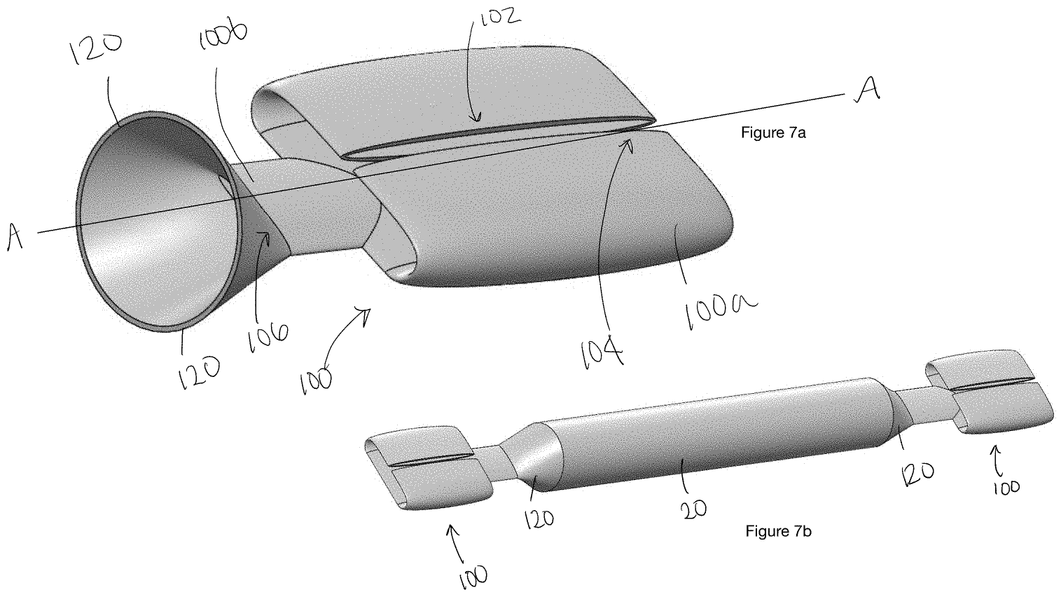

In one example, the connectors 100/200 are made from a flexible material that can be flattened into a low-profile mode for insertion into the artery. FIG. 7a shows the connector 100 in a low-profile mode. In the low-profile mode, the segments 100a/100b are substantially flattened to ease insertion of the connector 100 into the opening in artery 21. In some examples, the segments 100a/100b can be folded after being flattened. For instance, in the example of FIG. 7a, the ports 102/104 of segment 100a are folded towards the axis A1 so that the length of segment 100a is reduced by about half. The ease of insertion is particularly improved in examples where the portions 21a/21b of the artery are left at least partially connected to one another, discussed above, because the opening 50 can be made smaller since it does not need to accommodate the entire size of the connector 100. This in turn reduces the number of stitches/sutures required to eventually repair the artery 21 and improves healing and patient outcomes. The connector 100 can be expanded to the high profile operating mode, e.g., the mode shown in FIGS. 2a-b, with a spring-loaded or other mechanical feature in some examples, but in other examples, is configured to unfold and expand on its own when not being held in the low-profile operating mode by the user during insertion.

FIG. 7b shows the connectors 100 in the low profile mode and formed integrally with the AV graft as in the example of FIG. 6 discussed above.

Though FIGS. 7a-b show the connector 100, it should be understood that the same features are applicable to the connector 200.

Though the foregoing description is made with respect to the artery 21 for ease of reference, it should be understood that the same description applies to a vein 22 and its portions 22a/22b.

For a medical procedure such as hemodialysis, the AV graft 20 can be joined with the artery 21/vein 22 as discussed above. During the joining, the valve 24 is closed, e.g., the artery 21 and vein 22 are not fluidly connected. In some examples, the AV graft 20 can be charged with a biocompatible fluid such as oxygen or saline via the access ports 23 during the joining. After the joining, the biocompatible fluid can be drained from the AV graft 20 and the valve 24 can be opened, such as by any of the actuation mechanisms discussed in U.S. Pat. No. 10,610,633. When the valve 24 is opened, the artery 21 and vein 22 are fluidly connected during a hemodialysis procedure. When the procedure is complete, the valve 24 is closed and the AV graft 20 is re-charged with the biocompatible fluid. The AV graft 20, connectors 100/200, rings 110/210, and all other components discussed herein can be cleaned/sterilized in situ or after being explanted from the patient.

The preceding description is exemplary rather than limiting in nature. Variations and modifications to the disclosed examples may become apparent to those skilled in the art that do not necessarily depart from the essence of this disclosure. Thus, the scope of legal protection given to this disclosure can only be determined by studying the following claims.

What is claimed is:

1. A connector assembly for an arteriovenous (AV) graft, comprising:
a connector having a first segment arranged a long a first axis having first and second opposing ports and a second segment arranged along a second axis having a third port, each of the first and second ports configured to be joined to first and second portions of an artery or a vein, respectively; and
at least one ring configured to connect the first and second ports to the first and second portions of the artery or the vein by applying force about substantially the circumferential extent of the first segment of the connector and the first and second portions of the artery or the vein to join the first and second ports to the first and second portions of the artery or the vein, respectively.

2. The connector assembly of claim 1, wherein the ring includes first and second segments joined by a hinged connection.

3. The connector assembly of claim 1, wherein the at least one ring comprises a first ring at the first port and a second ring at the second port.

4. The connector assembly of claim 1, wherein the ring includes an opening configured to receive the second segment of the connector.

5. The connector assembly of claim 1, further comprising a seal arranged around at least one of the first, second, and third ports.

6. The connector assembly of claim 1, wherein an angle between the first and second axes is between 70 and 110 degrees.

7. The connector assembly of claim 6, wherein the first and second axes are perpendicular to one another.

8. The connector assembly of claim 1, wherein the connector is configured to be flattened into a low-profile operating mode.

9. The connector assembly of claim 1, further comprising a nozzle extending from the third port.

10. An arteriovenous (AV) graft assembly, comprising:
a first connector, the first connector having a first segment arranged a long a first axis having first and second opposing ports and a second segment arranged along a second axis having a third port, each of the first and second ports configured to be joined to first and second portions of an artery, respectively;
a second connector, the second connector having a first segment arranged a long a first axis having first and second opposing ports and a second segment arranged along a second axis having a third port, each of the first and second ports configured to be joined to first and second portions of a vein, respectively; and
an AV graft joined to the third port of each of the first and second connectors.

11. The AV graft assembly of claim 10, further comprising at least one ring configured to connect the first and second ports of the first and second connectors to the first and second portions of the artery or the vein by applying force about substantially the circumferential extent of the first segment of the connector and the first and second portions of the artery or the vein to join the first and second ports to the first and second portions of the artery or the vein, respectively.

12. The AV graft assembly of claim 10, wherein the AV graft is integral with at least one of the first and second connectors.

13. The AV graft assembly of claim 10, wherein at least one of the first and second connectors is configured to be flattened into a low-profile operating mode.

14. The AV graft assembly of claim 10, further comprising a nozzle extending from the third port of at least one of the first and second connectors.

15. A method of implanting a connector for an AV graft, comprising:
forming an opening in an artery or a vein between first and second portions of the artery or vein;
arranging a connector in the opening, the connector having a first segment arranged a long a first axis having first and second opposing ports and a second segment arranged along a second axis having a third port; and
joining the first and second portions of the artery or vein to the first and second ports.

16. The method of claim 15, wherein the step of forming the opening includes bisecting the artery or the vein.

17. The method of claim 15, wherein after the step of forming the opening, the first and second portions of the artery or the vein remain at least partially connected.

18. The method of claim 15, wherein joining the first and second ports to the first and second portions of the artery or vein, respectively, is accomplished by at least one ring.

19. The method of claim 18, further comprising flattening the connector into a low profile operating mode prior to the arranging.

20. The method of claim 16, wherein the AV graft includes a valve configured to control fluid flow through the AV graft, and further comprising the step of opening the valve.
